(19) United States
(12) Patent Application Publication
(10) Pub. No.: US 2016/0317841 A1
(43) Pub. Date: Nov. 3, 2016

(54) HUMAN SKIN TREATMENT TOOLS FOR HUMAN SKIN TREATMENT APPARATUS AND METHODS

(71) Applicant: Lifestyle Technologies Group Limited, Hertfordshire (GB)

(72) Inventors: Dean Nathanson, Hertfordshire (GB); Shilen Thakker, Hertfordshire (GB)

(21) Appl. No.: 15/143,826

(22) Filed: May 2, 2016

(30) Foreign Application Priority Data
Apr. 30, 2015 (GB) 1507487.5

(51) Int. Cl. A61N 7/00

(52) U.S. Cl. CPC A61N 7/00; A61N 2007/0034; A61N 2007/0056

(57) ABSTRACT

A human skin treatment tool includes a sleeve to be fitted to an ultrasonically excitable blade of a human skin treatment apparatus. The skin treatment tool has a skin treatment face configured to be vibrated against the skin by operation of a blade to treat the skin. The human skin treatment tool can be removed from the blade to allow the blade to be applied to the skin do perform different skin treatments and other human skin treatment tools having different skin treatment faces may fitted to the blade to apply yet more different skin treatments.

### FIELD OF THE INVENTION

The invention relates to human skin treatment tools for human skin treatment apparatus and methods.

### BACKGROUND TO THE INVENTION

A human skin treatment apparatus may make use of ultrasonic energy to cleanse the skin. A handheld human skin treatment apparatus may comprise a casing containing a control unit and an ultrasonic exciter and a blade that protrudes from the casing. When the ultrasonic exciter is energised it causes the blade to vibrate at high frequency. The edge of the vibrating blade may be applied to the skin to cleanse the skin. Alternatively, the flat surface of the vibrating blade may be applied to the skin to massage the skin and assist the skin to absorb moisture and nutrients applied to the skin.

### SUMMARY OF THE INVENTION

The invention provides a human skin treatment tool as specified in claim 1.

The invention also includes a human skin treatment apparatus as specified in claim 12.

The invention also includes a method of treating human skin as specified in claim 20.

### BRIEF DESCRIPTION OF THE DRAWINGS

In the disclosure that follows, reference will be made to the drawings in which:

FIG. 1 is a schematic side view of a human skin treatment apparatus;

FIG. 2 is schematic plan view of the human skin treatment apparatus of FIG. 1;

FIG. 3 is a schematic view of a human skin treatment tool fitted to the human skin treatment apparatus of FIG. 1;

FIG. 4 is a side view of the human skin treatment tool of FIG. 3;

FIG. 5 is a perspective view of the human skin treatment tool of FIG. 3;

FIG. 6 is a perspective view of another human skin treatment tool; and

FIG. 7 is a plan view of still another human skin treatment tool.

### DETAILED DESCRIPTION

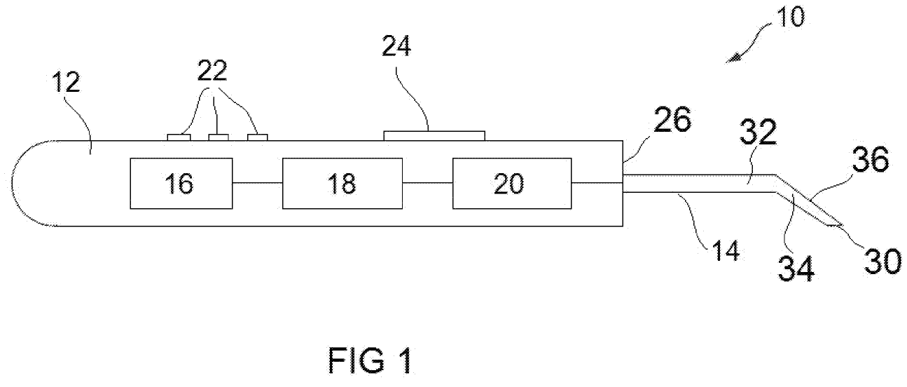

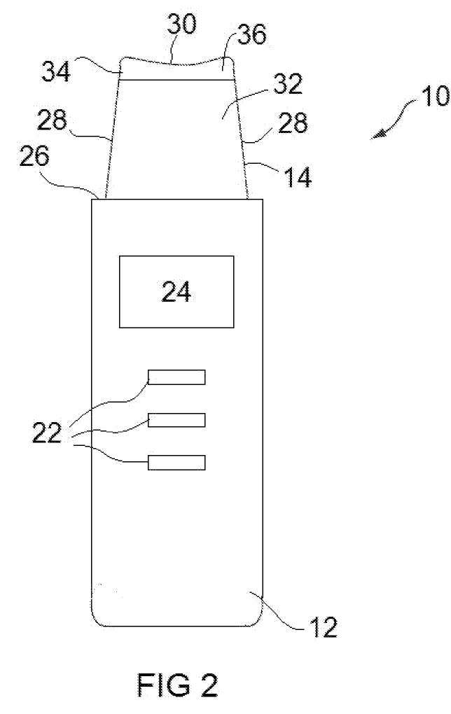

Referring to FIGS. 1 and 2, a human skin treatment apparatus 10 may comprise a handle 12 and a vibration member 14 projecting from the handle. The handle 12 may take the form of a casing. In the illustrated example the vibration member is in the form of a blade 14. The handle 12 may contain a power source 16, a control unit 18 and an ultrasonic exciter 20. The handle 12 may be provided with a user interface, which may comprise buttons 22 and a display 24. The buttons 22 may be configured to switch the skin treatment apparatus on/off and switch between operational modes. The display 24 may provide an indication of the currently selected operational mode, features associated with that mode, available modes and, where appropriate, the battery level. The display 24 may comprise an LCD or, in other less sophisticated examples, a series of lights. In other examples, the skin treatment apparatus 10 may have no display.

The power unit 16 may be a battery, which may be a rechargeable battery. If the power unit 16 comprises a rechargeable battery, the handle 12 may be provided with a socket (not shown) to connect with a transformer plug to facilitate charging from a source of mains electricity. Alternatively, the power unit 16 may be configured for contactless charging. Yet another alternative is for the skin treatment apparatus 10 to be mains powered, in which case the power unit 16 may comprise a transformer for conditioning incoming mains electricity. Yet another option is for the power lead to be provided with a socket comprising an integral transformer.

The control unit 18 is connected with the user interface 22, 24 and the ultrasonic exciter 20 to enable control of the operation of the ultrasonic exciter in response to operation of the buttons 22 and an indication of the operation mode on the display 24. The control unit 18 may comprise a chip or other suitable electronic circuitry.

The blade 14 is a relatively thin metal body that projects from an end 26 of the handle 12. The blade 14 may taper in the direction of extension away from the handle 12 such that its sides 28 converge towards a free end, or edge, 30. The free end 30 of the blade 14 may comprise a straight edge. Alternatively, the free end 30 may be profiled. For example, the free end 30 may curve inwardly as shown in the FIG. 2. The blade 14 may have a generally planar first portion 32 that extends from the handle 12. The first portion 32 may be generally coaxial with the handle. A second portion 34, which forms the free end of the blade, may be inclined with respect to the first portion 32. The blade 14 may, for example, be made of stainless steel.

The ultrasonic exciter 20, which may comprise a piezoelectric element, is directly or indirectly connected with the blade 14 such as to be able to cause high frequency, low amplitude, vibration of the blade. In some examples, the ultrasonic exciter 20 may cause the blade 14 to vibrate at a rate of 27,000 to 30,000 vibrations per second (vps). The control unit 18 may be operable to cause the ultrasonic exciter 20 to vibrate the blade 14 at different rates suited to different modes of operation so that a user can select a vibration mode for a desired treatment.

In use, the free end 30 of the blade 14 may be moved across the skin while being vibrated by the ultrasonic exciter 20. This mode of operation may push impurities to the surface and cause dead skin cells to become dislodged, thereby cleansing the skin. The rapid vibration of the blade 14 may also kill skin bacteria by causing the single cell walls of the bacteria cells to implode. In an alternative mode of operation, the blade 14 may be used to massage the skin. This can be done by applying the generally flat, or planar, front face 36 of the second end portion 32 of the blade 14 to the skin and pulling it over the skin while the blade is being vibrated by the ultrasonic exciter 20. This may cause more effective penetration of the skin by beneficial substances, such as moisturisers and nutrients applied to the skin prior to the massage treatment.

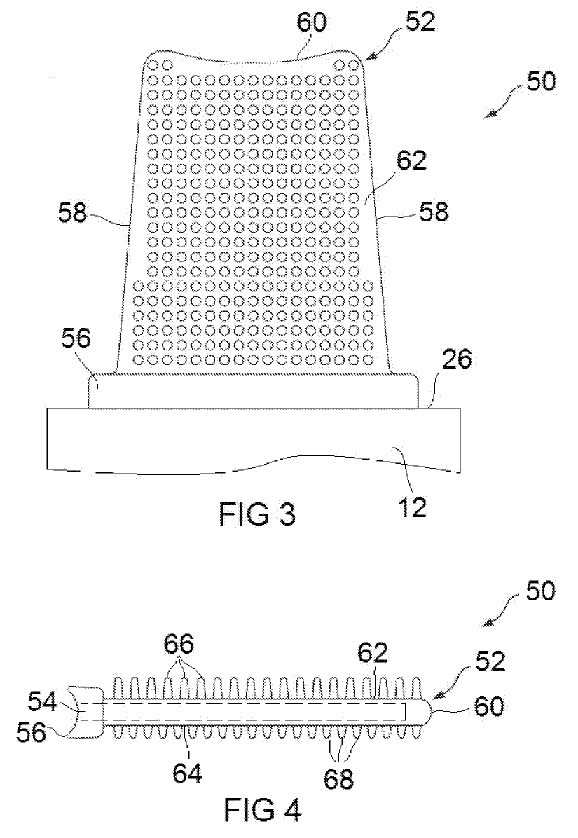

The human skin treatment apparatus 10 is also provided with at least one human skin treatment tool such as the skin treatment tools 50, 70 and 100 shown in FIGS. 3 to 6. The human skin treatment tools 50, 70, 100 can be fitted to the blade 14 to facilitate a particular skin treatment and removed when a different treatment is required or for cleaning.

Referring to FIGS. 3 to 5, the human skin treatment tool 50 comprises a sleeve 52 that can be slid onto the blade 14. The sleeve 52 defines a chamber, or recess, 54 that is open at a first end 56 of the sleeve. The chamber 54 is configured to receive the blade 14 and may be configured so that it at least substantially covers the blade up to the end 26 of the handle 12. The sleeve 52 may be made of an elastomeric material such as silicone rubber and configured so that the sleeve is retained on the blade 14 by at least one of its natural resilience and friction. The sleeve 52 has edges 58 that extend from the first end 56 to the opposite end 60 of the sleeve and opposed major sides 62, 64 that extend between and connect the ends 56, 60 and edges 58.

The skin treatment tool 50 comprises at least one skin treatment face. In the illustrated example, the skin treatment face is provided on the major side 62 and comprises a plurality of projections 66 projecting from a generally planar surface defined by the major side 62. While not essential, in the illustrated example the skin treatment face extends over at least substantially the entire surface area of the major side 62. The projections 66 may be upstanding elongate members whose longitudinal axes extend transverse to the plane of the major side 62. The projections 66 may be generally cylindrical rods. The projections 66 may have radiussed tips and in some examples may be conical. The projections 66 may be configured such that they are not rigid and can deflect under load. The skin treatment face of the sleeve 52 is configured for use in skin cleansing treatments. Optionally, a second skin treatment face may be provided on the major side 64. For, example, as shown in FIG. 4, the major side 64 may be provided with a plurality of projections 68 projecting from a generally planar surface defined by the major side 64. The projections 68 may be shorter or thicker than the projections 64 so as to be generally stiffer than the projections 64.

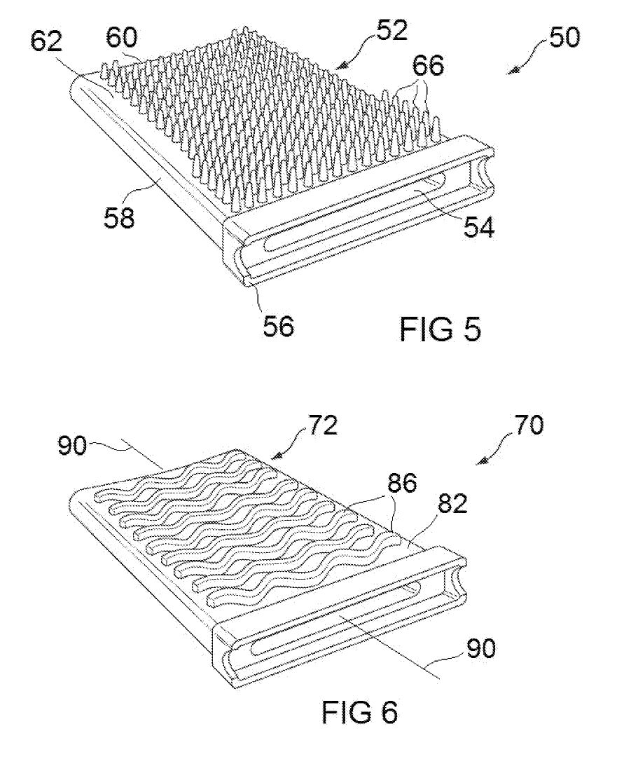

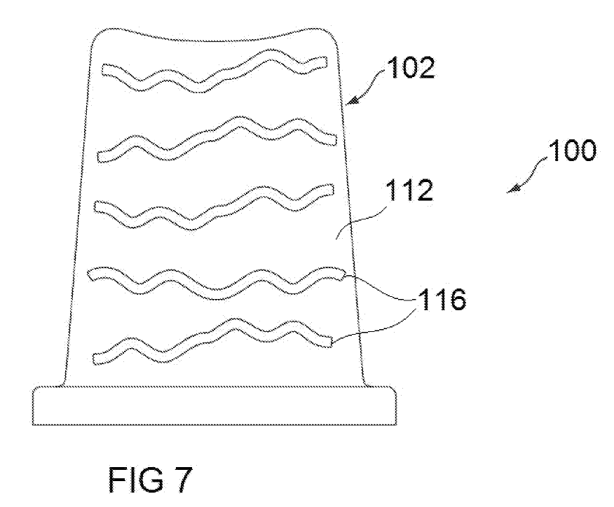

Although not essential, as best seen in FIGS. 4 and 5, the first end 56 of the sleeve may be enlarged to define a hood configured to fit over a mating formation (not shown) disposed at the end 26 of the handle 12. Advantageously, the hood may assist in preventing the ingress of liquids between the blade 14 and the handle 12. The hood may also be configured to make it easier for users to grip the skin treatment tool 50 while fitting it onto the blade 14.

The human skin treatment tool 70 is generally similar to the human skin treatment tool 50, the difference being in the configuration of the skin treatment face. To avoid unnecessary repetition of description, corresponding or similar features will be referenced by the same reference numerals incremented by 20. The skin treatment face of the human skin treatment tool 70 is provided on the major side 82 of the sleeve 72 and comprises a plurality of projections projecting from a generally planar surface defined by the major side 82. In this example the projections are ribs 86 that extend across the sleeve. The ribs 86 are spaced apart, but disposed relatively close together. The ribs 86 undulate in their lengthways direction and may be generally symmetric with respect to a longitudinal axis 90 of the sleeve 72. The ribs 86 may be arranged such that the peaks and troughs of adjacent waves are aligned in the lengthways direction of the sleeve 72. The ribs 86 may be of uniform height individually and collectively. The skin treatment face of the human skin treatment tool 70 is configured for skin treatments that improve skin circulation.

The human skin treatment tool 100 is generally similar to the skin treatment tool 50, the difference being in the configuration of the skin treatment face. To avoid unnecessary repetition of description, corresponding or similar features will be referenced by the same reference numerals incremented by 50. The human skin treatment tool 100 comprises a sleeve 102 that has a skin treatment face provided on the major side 112 of the sleeve. The skin treatment face comprises a plurality of projections 116 provided on a generally planar surface defined by the major side 112. In this example the projections comprise ribs 116 that extend across the sleeve 102. The ribs 116 may be irregularly spaced and non-symmetric. The ribs 116 may be taller than the ribs 66 and there may be fewer ribs 116 that are spread over the major side 112 such that the rib density is somewhat lower than the rib density of the skin treatment tool 70. The skin treatment face of the human skin treatment tool 100 is configured for skin massage treatments.

In each case, the human skin treatment tools 50, 70, 100 can be readily fitted onto the blade 14 to facilitate a particular skin treatment. Providing a human skin treatment apparatus with one or more human skin treatment tools facilitates the provision of an increased range of skin treatments using the same apparatus.

Examples of human skin treatment tools made of an elastomer, such as silicone, rubber can be stretched slightly to allow fitting and removal and when the stretch is released they may grip the blade by virtue of their natural resilience.

The human skin treatment tools may have only one skin treatment face. However, as shown in FIGS. 3 to 5, both major sides may be configured to provide respective skin treatment faces. The skin treatment faces may have the same configuration or different configurations to facilitate different skin treatments.

In the illustrated examples, the skin treatment faces are provided on a major side of the human skin treatment tool. In other examples, an edge of the skin treatment tool may be configured as a skin treatment face in addition to or as an alternative to a major side.

In the illustrated examples, the skin treatment faces extend over at least substantially the entire surface area of the major side of the human skin treatment tool. This is not essential and instead a skin treatment face may occupy just a part of a major side.

In the example shown in FIGS. 3 to 5, the upstanding projections lack rigidity so that they may deflect when in use. In other examples, a skin treatment face may be provided with upstanding projections that are configured so that they will not deflect significantly. The projections may for example take the form of small bumps or the like.

In the illustrated examples, the sleeves are shown with skin treatment faces comprising just one form of projection. In other examples, a skin treatment face may comprise more than one form of projection.

The human skin treatment tools may be provided with a human skin treatment apparatus or as separate items to be fitted to human skin treatment apparatus sold separately. Skin treatment tools may be sold as a kit comprising a plurality of skin treatment tools having different skin treatment faces. For example, a kit may comprise one each of the skin treatment tools 50, 70, 100.

In the illustrated examples the human skin treatment tools comprise a sleeve with an end provided with an opening to receive a vibration member of a human skin treatment apparatus and an opposite end that is closed. The sleeve can be fitted onto the vibration member like a glove and may cover the entire part of the vibration member that projects from the handle. In other examples, the sleeve may cover only a portion of the vibration member starting at its free end so that a portion of the vibration member between the handle and sleeve is exposed. In still other examples, the sleeve may be open at both ends and may for example be configured so that it can fit onto the vibration member while leaving the free end of the vibration member exposed.

In the example illustrated by FIGS. 1 and 2, the human skin treatment apparatus is a handheld device with a control unit and user interface mounted in or on the handle. In other examples, one or both of a control unit and parts or all of a user interface may be provided as separate unit and the handle may be solid.

1. A human skin treatment tool comprising:
a sleeve to be removably fitted to a blade of a human skin treatment apparatus comprising an exciter configured to cause ultrasonic vibration of said blade,
wherein said sleeve has a skin treatment face configured to treat the skin when, in use, it is vibrated against the skin by operation of said human skin treatment apparatus.

2. A human skin treatment tool as claimed in claim 1, wherein said sleeve is made of an elastomeric material.

3. A human skin treatment tool as claimed in claim 2, wherein said elastomeric material is silicone rubber.

4. A human skin treatment tool as claimed in claim 1, wherein said skin treatment face comprises a surface provided with a plurality of projections.

5. A human skin treatment tool as claimed in claim 4, wherein said projections comprise ribs extending across said skin treatment face.

6. A human skin treatment tool as claimed in claim 4, wherein said projections comprise members each having a longitudinal axis that extends transverse to said surface.

7. A human skin treatment tool as claimed in claim 4, wherein said projections are deflectable with respect to said surface.

8. A human skin treatment tool as claimed in claim 1, wherein said sleeve comprises opposed major sides and said skin treatment face is provided on a said major side.

9. A human skin treatment tool as claimed in claim 1, wherein said sleeve comprises a planar body defining a recess to receive said blade.

10. A human skin treatment tool as claimed in claim 9, wherein said body is a fruto-conical body.

11. A human skin treatment tool as claimed in claim 9, wherein said recess extends inwardly from an end of said body that is provided with an enlargement defining a hood to receive a projection provided on an end of said human skin treatment apparatus.

12. A human skin treatment apparatus comprising:
a handle;
a blade projecting from said handle;
an exciter operable to cause ultrasonic vibration of said blade to enable skin exfoliation by said blade; and
a removable human skin treatment tool to be fitted to said blade,
wherein said human skin treatment tool comprises a sleeve having a skin treatment face configured to be vibrated against human skin by vibration of said blade to treat said human skin.

13. A human skin treatment apparatus as claimed in claim 12, wherein said blade has a first end portion connected with said exciter and a free end portion extending from and angled with respect to said first end portion.

14. A human skin treatment apparatus as claimed in claim 12, wherein said sleeve is made of an elastomeric material.

15. A human skin treatment tool as claimed in claim 12, wherein said skin treatment face comprises a surface provided with a plurality of projections.

16. A human skin treatment tool as claimed in claim 15, wherein said projections comprise ribs extending across said skin treatment face.

17. A human skin treatment tool as claimed in claim 15, wherein said projections comprise members each having a longitudinal axis that extends transverse to said surface.

18. A human skin treatment tool as claimed in claim 15, wherein said projections are deflectable with respect to said surface.

19. A human skin treatment tool as claimed in claim 12, wherein said sleeve comprises opposed major sides and said skin treatment face is provided on a said major side.

20. A method of treating human skin, said method comprising:
selecting a human skin treatment tool that has a skin treatment face;
fitting said human skin treatment tool to a blade of a human skin treatment apparatus that comprises an exciter configured to cause ultrasonic vibration of said blade;
causing said exciter to vibrate said blade; and
applying said skin treatment face to human skin to treat said skin.

21. A method of treating human skin as claimed in claim 20, comprising fitting said human skin treatment tool to said blade by stretching said skin treatment tool prior to inserting said blade into said human skin treatment tool and permitting said skin treatment tool to resile to grip the inserted blade.
